(19) United States
(12) Patent Application Publication
(10) Pub. No.: US 2015/0190077 A1
(43) Pub. Date: Jul. 9, 2015

(54) ELECTRONIC DEVICE AND PHOTOPLETHYSMOGRAPHY METHOD

(71) Applicant: Samsung Electronics Co., Ltd., Suwon-si (KR)

(72) Inventors: Jongjin KIM, Hwaseong-si (KR); Jongmin CHOI, Seoul (KR); Jooman HAN, Seoul (KR)

(21) Appl. No.: 14/586,029

(22) Filed: Dec. 30, 2014

Related U.S. Application Data
(60) Provisional application No. 61/924,533, filed on Jan. 7, 2014.

(30) Foreign Application Priority Data
Mar. 28, 2014 (KR) 10-2014--0036648

(51) Int. Cl. A61B 5/1455; A61B 5/103; A61B 5/00; A61B 5/0205

(52) U.S. Cl. CPC A61B 5/14551; A61B 5/02055; A61B 5/1032; A61B 5/7207; A61B 5/7221; A61B 5/02416

(57) ABSTRACT

A photoplethysmography (PPG) method is provided. The PPG method includes performing at least one of PPG signal measurement and surrounding environment information sensing, and determining a characteristic of a light source for measuring a PPG signal based on at least one of the measured PPG signal or the sensed surrounding environment information.

### CROSS-REFERENCE TO RELATED APPLICATION(S)

This application claims the benefit under 35 U.S.C. §119(e) of a U.S. Provisional application filed on Jan. 7, 2014 in the U.S. Patent and Trademark Office and assigned Ser. No. 61/924,533 and under 35 U.S.C. §119(a) of a Korean patent application filed on Mar. 28, 2014 in the Korean Intellectual Property Office and assigned Serial No. 10-2014-0036648, the entire disclosure of each of which is hereby incorporated by reference.

### TECHNICAL FIELD

The present disclosure relates to a method of measuring photoplethysmography (PPG) by using a plurality of light sources, and an electronic device implementing the same.

### BACKGROUND

Recently, based on the development of communication and related technology, applications with various functions are downloaded and used in an electronic device, for example, a portable terminal, including a smart phone. Accordingly, the portable terminal has been developed so as to provide various functions which may be usefully used in the life of a user in general, as well as existing call and message functions.

Photoplethysmography (PPG) is a technology of measuring a heart rate, oxygen saturation, or the like at a body part, such as a fingertip, in which an artery passes, by using light. Various cardiovascular related health information, such as a heart rate or and oxygen saturation, is calculated by using the PPG technology.

Recently, a technology for enabling a user to conveniently obtain his/her health information by providing a PPG function by a portable terminal has been developed.

The above information is presented as background information only to assist with an understanding of the present disclosure. No determination has been made, and no assertion is made, as to whether any of the above might be applicable as prior art with regard to the present disclosure.

### SUMMARY

During the photoplethysmography (PPG) using an electronic device, various factors may influence generation of a PPG signal. For example, a signal noise caused by a movement of a user, a skin color of a user, a surrounding temperature, a surrounding light, and the like may influence the generation of a PPG signal. Further, PPG signals may be detected in various ranges by an individual characteristic of each person. Further, a factor, which may influence the PPG, may be considered according to the type of information which is desired to be obtained by using the PPG signal, that is, an object of the PPG.

Aspects of the present disclosure are to address at least the above-mentioned problems and/or disadvantages and to provide at least the advantages described below. Accordingly, an aspect of the present disclosure is to provide an electronic device capable of more accurately generating a PPG signal in order to obtain biometric numerical value information desired by a user by considering various influences, and a PPG method.

In accordance with an aspect of the present disclosure, a PPG method is provided. The PPG method includes performing at least one of PPG signal measurement and surrounding environment information sensing, and determining a characteristic of a light source for measuring a PPG signal based on at least one of the measured PPG signal or the sensed surrounding environment information.

In accordance with another aspect of the present disclosure, an electronic device is provided. The electronic device includes a plurality of light sources having different wavelength characteristics, a PPG signal detection unit configured to detect light, which is irradiated from at least one of the plurality of light sources, and passes through or is reflected from a user, and a controller configured to detect a PPG signal from an output signal of the PPG signal detection unit, and to select at least one of the plurality of light sources based on the detected PPG signal.

In accordance with another aspect of the present disclosure, a PPG method is provided. The PPG method includes a computer readable recording medium, in which a program for executing an operation performing at least one of PPG signal measurement and surrounding environment information sensing, and determining a characteristic of a light source for the PPG signal measurement based on at least one of the measured PPG signal and the sensed surrounding environment information is recorded.

According to various embodiments of the present disclosure, the PPG method may determine a characteristic of a light source for measuring a PPG signal by considering various factors influencing generation of the PPG signal.

Further, according to various embodiments of the present disclosure, the electronic device may perform PPG by selecting the most appropriate light source, thereby improving accuracy in measurement of cardiovascular related health information.

Other aspects, advantages, and salient features of the disclosure will become apparent to those skilled in the art from the following detailed description, which, taken in conjunction with the annexed drawings, discloses various embodiments of the present disclosure.

### BRIEF DESCRIPTION OF THE DRAWINGS

The above and other objects, features, and advantages of certain embodiments of the present disclosure will be more apparent from the following description taken in conjunction with the accompanying drawings, in which:

FIG. 1 illustrates a network environment including an electronic device according to an embodiment of the present disclosure;

FIG. 2 is a block diagram illustrating an electronic device according to an embodiment of the present disclosure;

FIG. 3 illustrates a waveform of a photoplethysmography (PPG) signal according to an embodiment of the present disclosure;

FIG. 4 is a diagram schematically illustrating a PPG measurement module of the electronic device according to an embodiment of the present disclosure;

FIG. 5 is a flowchart schematically illustrating a PPG method according to an embodiment of the present disclosure;

FIG. 6 is a flowchart illustrating an example, in which a measurement mode and a measured PPG signal are considered in the PPG method, according to an embodiment of the present disclosure; and

FIG. 7 is a flowchart illustrating an example, in which neighboring environment information is considered in the PPG method, according to an embodiment of the present disclosure.

Throughout the drawings, it should be noted that like reference numbers are used to depict the same or similar elements, features, and structures.

### DETAILED DESCRIPTION

The following description with reference to the accompanying drawings is provided to assist in a comprehensive understanding of various embodiments of the present disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the present disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the present disclosure. Accordingly, it should be apparent to those skilled in the art that the following description of various embodiments of the present disclosure is provided for illustration purpose only and not for the purpose of limiting the present disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

In the present disclosure, the expression “include” or “may include” refers to existence of a corresponding function, operation, or element, and does not limit one or more additional functions, operations, or elements. In the description, it should be understood that the terms “include” or “have” indicate existence of a feature, a number, a step, an operation, a structural element, parts, or a combination thereof, and do not previously exclude the existences or probability of addition of one or more another features, numeral, steps, operations, structural elements, parts, or combinations thereof.

The expression such as “or” or the like in various embodiments of the present disclosure include any and all of the combinations of words disclosed together. For example, the expression “A or B” may include A, may include B, or may include both A and B.

The expression “1”, “2”, “first”, or “second” used in various embodiments of the present disclosure may modify various components of various embodiments but does not limit the corresponding components. For example, the above expressions do not limit the sequence and/or importance of the elements. The above expressions are used merely for the purpose to distinguish an element from the other elements. For example, a first user device and a second user device indicate different user devices although both of them are user devices. For example, without departing from the scope of the present disclosure, a first structural element may be named a second structural element. Similarly, the second structural element also may be named the first structural element.

In the case where a component is referred to as being “connected” or “accessed” to another component, it should be understood that not only the component is directly connected or accessed to the other component, but also there may exist another component between them. Meanwhile, in the case where a component is referred to as being “directly connected” or “directly accessed” to another component, it should be understood that there is no component therebetween.

In the present disclosure, the terms are used to describe a specific embodiment, and are not intended to limit the present disclosure.

Unless defined differently, all terms used herein, which include technical terminologies or scientific terminologies, have the same meaning as that understood by a person skilled in the art to which the present disclosure belongs. Such terms as those defined in a generally used dictionary are to be interpreted to have the meanings equal to the contextual meanings in the relevant field of art, and are not to be interpreted to have ideal or excessively formal meanings unless clearly defined in the present disclosure.

For example, the electronic device may include at least one of a smartphone, a tablet Personal Computer (PC), a mobile phone, a video phone, an electronic book (e-book) reader, a desktop PC, a laptop PC, a netbook computer, a Personal Digital Assistant (PDA), a Portable Multimedia Player (PMP), an MP3 player, a mobile medical appliance, a camera, and a wearable device (e.g. a Head-Mounted-Device (HMD) such as electronic glasses, electronic clothes, an electronic bracelet, an electronic necklace, an electronic appcessory, electronic tattoos, or a smartwatch).

According to various embodiments of the present disclosure, the electronic device may be a smart home appliance. The smart home appliance as an example of the electronic device may include at least one of, for example, a television (TV), a Digital Video Disk (DVD) player, an audio, a refrigerator, an air conditioner, a vacuum cleaner, an oven, a microwave oven, a washing machine, an air cleaner, a set-top box, a TV box (e.g., Samsung HomeSync™, Apple TV™, or Google TV™), a game console, an electronic dictionary, an electronic key, a camcorder, and an electronic picture frame.

According to an embodiment of the present disclosure, an electronic device may include at least one of various medical devices such as a magnetic resonance angiography (MRA) scanner, a magnetic resonance imaging (MRI) scanner, a computed tomography (CT) scanner, a scanner, an ultrasonograph, or the like, a navigation device, a Global Positioning System (GPS) receiver, an Event Data Recorder (EDR), a Flight Data Recorder (FDR), a vehicle infotainment device, an electronic equipment for ship (for example a ship navigation device and gyro-compass and the like, avionics, a security device, a head unit for a vehicle, an industrial or household robot, an automatic teller machine (ATM) in a banking facility or a point of sale (POS) in a store.

According to various embodiments of the present disclosure, the electronic device may include at least one of a part of furniture or a building/structure, an electronic board, an electronic signature receiving device, a projector, and various kinds of measuring instruments (e.g., a water meter, an electric meter, a gas meter, or a radio wave meter). Electronic devices according to various embodiments of the present disclosure may be one or more combinations of the above-described devices. Also, the electronic device according to the present disclosure may be a flexible device. Further, it is obvious to those skilled in the art that electronic devices according to various embodiments of the present disclosure are not limited to the above-described devices.

Hereinafter, an electronic device according to various embodiments of the present disclosure will be described with reference to the accompanying drawings. The term “a user” used in various embodiments of the present disclosure may refer to a person who uses electronic devices or a device (e.g., an artificial intelligence electronic device) that uses electronic devices.

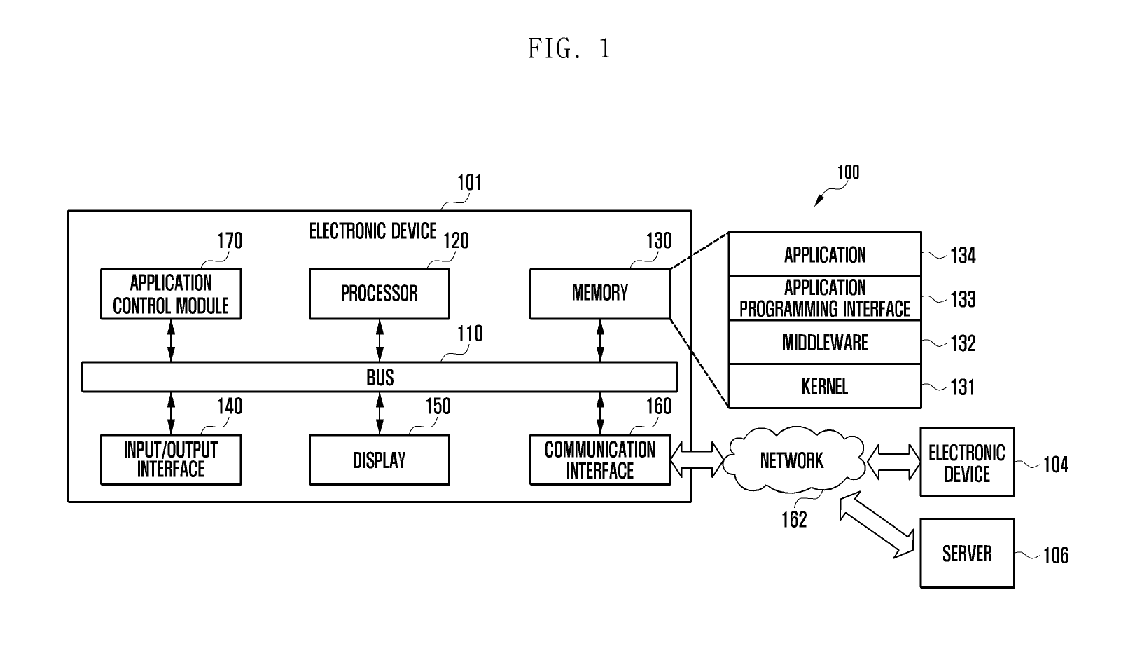

FIG. 1 illustrates a network environment including an electronic device according to various embodiments of the present disclosure.

Referring to FIG. 1, the network environment 100 includes the electronic device 101 that may include a bus 110, a processor 120, a memory 130, an input/output interface 140, a display 150, a communication interface 160, and an application configuring module 170.

The bus 110 may be a circuit for connecting the above-described elements with each other and transferring communication (for example, a control message) between the above-described elements.

The processor 120 may receive instructions from any or all of the aforementioned elements (for example, the memory 130, the input/output interface 140, the display 150, the communication interface 160, and the application configuring module 170) through the bus 110, decipher the received instructions, and perform a calculation or data processing according to the deciphered instructions.

The memory 130 may store instructions or data received from the processor 120 or other elements (for example, the input/output interface 140, the display 150, the communication interface 160, the message management module 170, or the like), or generated by the processor 120 or other elements. The memory 130 may include programming modules, such as, a kernel 131, a middleware 132, an application programming interface (API) 133, or application(s) 134. Each of the programming modules described above may be configured by software, firmware, hardware, or a combination of two or more thereof.

The kernel 131 may control or manage system resources (for example, the bus 110, the processor 120, the memory 130, or the like) which are used in performing operations or functions implemented by other programming modules, for example the middleware 132, the API 133, or the application(s) 134. Furthermore, the kernel 131 may provide an interface through which the middleware 132, the API 133, and the application(s) 134 may access individual elements of the electronic device 101 to control or manage the individual elements.

The middleware 132 may serve as an intermediary such that the API 133 or the application(s) 134 communicates with the kernel 131 to transceive data. Furthermore, in connection with task requests received from the application(s) 134, the middleware 132 may perform a control (for example, scheduling or load balancing) for the task requests using, for example, a method of assigning a priority for using the system resources (for example, the bus 110, the processor 120, and the memory 130) of the electronic device 101 to at least one of the application(s) 134.

The API 133 is an interface by which the application(s) 134 controls functions provided from the kernel 131 or the middleware 132, and may include, for example, at least one interface or function (for example, instructions) for file control, window control, image processing, or text control.

According to various embodiments of the present disclosure, the application(s) 134 may include a Short Message Service (SMS)/Multimedia Message Service (MMS) application, an e-mail application, a calendar application, an alarm application, a health care application (for example, an application for measuring a work rate or a blood sugar), an environment information application (for example, an application for providing atmospheric pressure, humidity, or temperature information). Additionally or alternatively, the application(s) 134 may be an application related to an exchange of information between the electronic device 101 and an external electronic device (for example, an electronic device 104). The application associated with the exchange of information may include, for example, a notification relay application for transferring specific information to the external electronic device or a device management application for managing the external electronic device.

For example, the notification relay application may include a function of relaying notification information created by other applications of the electronic device 101 (for example, the SMS/MMS application, the e-mail application, the health care application, and the environment information application) to the external electronic device (for example, the electronic device 104). Additionally or alternatively, the notification relay application may receive notification information from, for example, an external electronic device (for example, the electronic device 104) and provide the received notification information to a user. For example, the device management application may manage (for example, install, delete, or update) a function for at least some parts of the external electronic device (for example, the electronic device 104), communicating with the electronic device 101 (for example, a function of turning on/off the external electronic device itself (or some components) or a function of adjusting luminance (or resolution) of the display), applications operating in the external electronic device, or services provided by the external electronic device (for example, a call service and a message service).

According to various embodiments of the present disclosure, the application(s) 134 may include applications, which are designated according to the property (for example, the type of electronic device) of the external electronic device (for example, the electronic device 104). For example, in a case where the external electronic device is an MP3 player, the application(s) 134 may include an application related to the reproduction of music. Similarly, in a case where the external electronic device is a mobile medical appliance, the application(s) 134 may include an application related to health care. According to an embodiment of the present disclosure, the application(s) 134 may include at least one of applications designated in the electronic device 101 or applications received from external electronic devices (for example, a server 106, or the electronic device 104).

The input/output interface 140 may transfer instructions or data input from a user through an input/output device (for example, a sensor, a keyboard, or a touch screen) to, for example, the processor 120, the memory 130, the communication interface 160, or the application configuring module 170 through the bus 110. For example, the input/output interface 140 may provide data for a user's touch, which is input through the touch screen, to the processor 120. Furthermore, the input/output interface 140 may output instructions or data, which are received from the processor 120, the memory 130, the communication interface 160, or the application configuring module 170 through the bus 110, through the input/output device (for example, a speaker or a display). For example, the input/output interface 140 may output voice data processed through the processor 120 to a user through a speaker.

The display 150 may display various information (for example, multimedia data or text data) to a user.

The communication interface 160 may establish communication between the electronic device 101 and the external electronic device (for example, the electronic device 104 or the server 106). For example, the communication interface 160 may be connected to a network 162 through wireless or wired communication to communicate with the external device. The wireless communication may include at least one of, for example, Wireless Fidelity (Wi-Fi), Bluetooth (BT), Near Field Communication (NFC), a Global Positioning System (GPS), and cellular communication (for example, Long Term Evolution (LTE), Long Term Evolution-Advanced (LTE-A), Code Division Multiple Access (CDMA), Wideband CDMA (WCDMA), Universal Mobile Telecommunication System (UMTS), Wireless Broadband (WiBro), or Global System for Mobile communication (GSM)). The wired communication may include at least one of a Universal Serial Bus (USB), a High Definition Multimedia Interface (HDMI), Recommended Standard 232 (RS-232), and a Plain Old Telephone Service (POTS).

According to an embodiment of the present disclosure, the network 162 may be a telecommunication network. The communication network may include at least one of a computer network, the Internet, the Internet of things, and a telephone network. According to an embodiment of the present disclosure, a protocol (for example, a transport lay protocol, data link layer protocol, or a physical layer protocol) for communication between the electronic device 101 and the external device may be supported by at least one of the application(s) 134, the application programming interface 133, the middleware 132, the kernel 131, and the communication interface 160.

The application configuring module 170 may process at least some pieces of information acquired from other elements (for example, the processor 120, the memory 130, the input/output interface 140, and the communication interface 160), and may provide the processed information to a user through various methods. For example, the application control module 170 may recognize information on a connected component included in the electronic device 101, store the information on the connected component in the memory 130, and execute the application(s) 134 based on the information about the connected component.

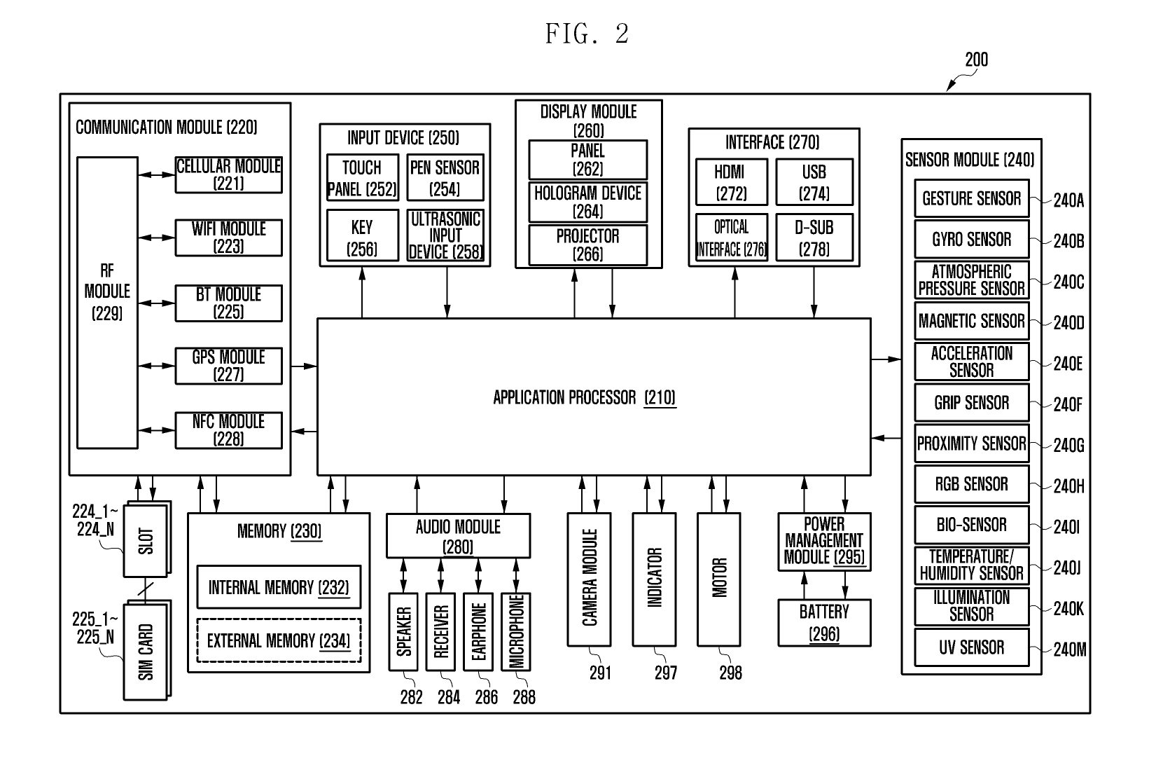

FIG. 2 is a block diagram illustrating an electronic device according to an embodiment of the present disclosure. The electronic device of FIG. 2 may constitute, for example, all or a part of the electronic device 101 shown in FIG. 1.

Referring to FIG. 2, the electronic device 200 may include at least one Application Processor (AP) 210, a communication module 220, one or more Subscriber Identification Module (SIM) cards 225_1 to 225_N, a memory 230, a sensor module 240, an input device 250, a display module 260, an interface 270, an audio module 280, a camera module 291, a power management module 295, a battery 296, an indicator 297, and a motor 298.

The AP 210 may control a plurality of hardware or software components connected to the AP 210 by driving an operating system or an application program, process various data including multimedia data, and perform calculations. The AP 210 may be implemented by, for example, a System on Chip (SoC). According to an embodiment of the present disclosure, the AP 210 may further include a Graphic Processing Unit (GPU).

The communication module 220 (for example, the communication interface 160) may perform data transmission/reception in communication between the electronic device 200 (for example, the electronic device 101) and other electronic devices (for example, the electronic device 104 and the server 106) connected thereto through a network. According to an embodiment of the present disclosure, the communication module 220 may include a cellular module 221, a Wi-Fi module 223, a BT module 225, a GPS module 227, an NFC module 228, and a Radio Frequency (RF) module 229.

The cellular module 221 may provide a voice call, a video call, a message service, or an Internet service through a communication network (for example, LTE, LTE-A, CDMA, WCDMA, UMTS, WiBro, GSM, or the like). Further, the cellular module 221 may distinguish and authenticate electronic devices within a communication network by using a subscriber identification module (for example, using one or more of the SIM cards 225_1 to 225_N). According to an embodiment of the present disclosure, the cellular module 221 may perform at least some of the functions that the AP 210 may provide. For example, the cellular module 221 may perform at least a multimedia control function.

According to an embodiment of the present disclosure, the cellular module 221 may include a Communication Processor (CP). Further, the cellular module 221 may be implemented as, for example, an SoC. It is illustrated that elements, such as the cellular module 221 (for example, a communication processor), the memory 230, or the power management module 295 are separate elements from the AP 210, but according to an embodiment of the present disclosure, the AP 210 may be implemented to include at least some (for example, the cellular module 221) of the aforementioned elements.

According to an embodiment of the present disclosure, the AP 210 or the cellular module 221 (for example, the communication processor) may load instructions or data received from at least one of a non-volatile memory and other elements connected thereto to a volatile memory, and may process the loaded instructions or data. Further, the AP 210 or the cellular module 221 may store data received from or generated by at least one of other elements in a non-volatile memory.

The Wi-Fi module 223, the BT module 225, the GPS module 227, and the NFC module 228 may include, for example, a processor for processing data transmitted/received through the corresponding module. It is illustrated that the cellular module 221, the WiFi module 223, the BT module 225, the GPS module 227, or the NFC module 228 are separate blocks, but according to an embodiment of the present disclosure, at least one (for example, two or more) of the cellular module 221, the WiFi module 223, the BT module 225, the GPS module 227, and the NFC module 228 may be included in one Integrated Chip (IC) or an IC package. For example, at least some (for example, a communication processor corresponding to the cellular module 221 and a Wi-Fi processor corresponding to the Wi-Fi module 223) processors corresponding to the cellular module 221, the Wi-Fi module 223, the BT module 225, the GPS module 227, and the NFC module 228, respectively, may be implemented as one SoC.

The RF module 229 may transceive data, for example, an RF signal.

Although not illustrated, the RF unit 229 may include, for example, a transceiver, a Power Amp Module (PAM), a frequency filter, a Low Noise Amplifier (LNA), or the like. Furthermore, the RF module 229 may further include a component, such as a conductor or a conducting wire, for transmitting/receiving electronic waves over a free air space in wireless communication. It is illustrated that the cellular module 221, the WiFi module 223, the BT module 225, the GPS module 227, and the NFC module 228 share one RF module 229, but according to an embodiment of the present disclosure, at least one of the cellular module 221, the WiFi module 223, the BT module 225, the GPS module 227, and the NFC module 228 may transceive an RF signal through a separate RF module.

The SIM cards 225_1 to 225_N may be cards included in the subscriber identification module, and be inserted to a slot 224_1 to 224_N formed at a specific position of the electronic device. The SIM cards 225_1 to 225_N may include unique identification information (for example, Integrated Circuit Card IDentifier (ICCID)) or subscriber information (for example, International Mobile Subscriber Identity (IMSI)).

The memory 230 (for example, the memory 130) may include an internal memory 232 or an external memory 234. The internal memory 232 may include at least one of a volatile memory (for example, a Dynamic Random Access Memory (DRAM), a Static RAM (SRAM), a Synchronous Dynamic RAM (SDRAM), and the like) and a non-volatile memory (for example, a One Time Programmable Read Only Memory (OTPROM), a Programmable ROM (PROM), an Erasable and Programmable ROM (EPROM), an Electrically Erasable and Programmable ROM (EEPROM), a mask ROM, a flash ROM, a Not And (NAND) flash memory, a Not Or (NOR) flash memory, and the like).

According to an embodiment of the present disclosure, the internal memory 232 may be a Solid State Drive (SSD). The external memory 234 may further include a flash drive, for example, a Compact Flash (CF), a Secure Digital (SD), a Micro Secure Digital (Micro-SD), a Mini Secure Digital (Mini-SD), an extreme Digital (xD), or a memory stick. The external memory 234 may be functionally connected to the electronic device 200 through various interfaces. According to an embodiment of the present disclosure, the electronic device 200 may further include a storage device (or storage medium), such as a hard disk drive.

The sensor module 240 may measure a physical quantity or detect an operation state of the electronic device 200, and may convert the measured or detected information to an electronic signal. The sensor module 240 may include at least one of, for example, a gesture sensor 240A, a gyro sensor 240B, an atmospheric pressure sensor 240C, a magnetic sensor 240D, an acceleration sensor 240E, a grip sensor 240F, a proximity sensor 240G, a color sensor 240H (for example, a Red/Green/Blue (RGB) sensor), a bio-sensor 240I, a temperature/humidity sensor 240J, an illumination sensor 240K, and an Ultra Violet (UV) sensor 240M. Additionally or alternatively, the sensor module 240 may include, for example, an E-nose sensor (not illustrated), an electromyography (EMG) sensor (not illustrated), an electroencephalogram (EEG) sensor (not illustrated), an electrocardiogram (ECG) sensor (not illustrated), an Infrared (IR) sensor, an iris sensor (not illustrated), or a fingerprint sensor. The sensor module 240 may further include a control circuit for controlling one or more sensors included therein.

The input device 250 may include a touch panel 252, a (digital) pen sensor 254, a key 256, or an ultrasonic input device 258. The touch panel 252 may recognize a touch input through at least one of, for example, a capacitive type, a resistive type, an infrared type, an electromagnetic induction type, and an acoustic wave type. The touch panel 252 may further include a control circuit. In the case of the capacitive type or the electromagnetic induction type, a physical contact input or a proximity input is allowed. The touch panel 252 may also further include a tactile layer. In this case, the touch panel 252 may provide a tactile reaction to a user.

The (digital) pen sensor 254 may be implemented, for example, using a method identical or similar to a method of receiving a touch input of a user, or using a separate sheet for recognition. The key 256 may include, for example, a physical button, an optical key, or a keypad. The ultrasonic input device 258 is a device that may detect an acoustic wave by a microphone (for example, a microphone 288) of the electronic device 200 through an input tool generating an ultrasonic signal to identify data, and may perform wireless recognition. According to an embodiment of the present disclosure, the electronic device 200 may also receive a user input from an external device (for example, a computer or a server) connected thereto, using the communication module 220.

The display module 260 (for example, the display 150) may include a panel 262, a hologram device 264, or a projector 266. The panel 262 may be, for example, a Liquid Crystal Display (LCD) or an Active Matrix Organic Light Emitting Diode (AM-OLED). The panel 262 may be implemented to be, for example, flexible, transparent, or wearable. The panel 262 may also be configured as one module together with the touch panel 252. The hologram device 264 may show a stereoscopic image in the air using interference of light. The projector 266 may project light onto a screen to display an image. For example, the screen may be located inside or outside the electronic device 200. According to an embodiment of the present disclosure, the display module 260 may further include a control circuit for controlling the panel 262, the hologram device 264, or the projector 266.

The interface 270 may include, for example, an HDMI 272, a USB 274, an optical interface 276, or a D-subminiature (D-sub) 278. The interface 270 may be included in, for example, the communication interface 160 illustrated in FIG. 1. Additionally or alternatively, the interface 270 may include, for example, a Mobile High-definition Link (MHL) interface, an SD card/Multi-Media Card (MMC) interface, or an Infrared Data Association (IrDA) standard interface.

The audio module 280 may bilaterally convert a sound and an electronic signal. At least some elements of the audio module 280 may be included in, for example, the input/output interface 140 illustrated in FIG. 1. The audio module 280 may process voice information input or output through, for example, a speaker 282, a receiver 284, earphones 286, or the microphone 288.

The camera module 291 is a device for capturing a still image or a video, and, according to an embodiment of the present disclosure, may include one or more image sensors (for example, a front side sensor or a back side sensor), a lens, an Image Signal Processor (ISP) (not illustrated), or a flash (not illustrated) (for example, an LED or xenon lamp).

The power management module 295 may manage power of the electronic device 200. Although not illustrated, the power management module 295 may include, for example, a Power Management Integrated Circuit (PMIC), a charger IC, or a battery or fuel gauge.

The PMIC may be mounted in, for example, an integrated circuit or an SoC semiconductor. Charging methods may be classified into a wired charging method and a wireless charging method. The charger IC may charge a battery and prevent over voltage or over current from flowing from a charger. According to an embodiment of the present disclosure, the charger IC may include a charger IC for at least one of the wired charging method and the wireless charging method. Examples of the wireless charging method include a magnetic resonance scheme, a magnetic induction scheme, an electromagnetic scheme, and an additional circuit for wireless charging, such as a coil loop circuit, a resonance circuit, or a rectifier circuit, may be added.

The battery fuel gauge may measure, for example, the remaining capacity of the battery, a charging voltage and current, or temperature of the battery 296. The battery 296 may store or generate electricity, and may supply power to the electronic device 200 by using the stored or generated electricity. The battery 296 may include, for example, a rechargeable battery or a solar battery.

The indicator 297 may display a particular status of the electronic device 200 or a part thereof (for example, the AP 210), for example, a booting status, a message status, or a charging status. The motor 298 may convert an electrical signal to a mechanical vibration. Although not illustrated, the electronic device 200 may include a processing unit (for example, GPU) for mobile TV support. The processing unit for supporting a mobile TV may process media data according to a standard of Digital Multimedia Broadcasting (DMB), Digital Video Broadcasting (DVB), media flow, or the like.

The aforementioned elements of the electronic device according to various embodiments of the present disclosure may be configured with one or more components, and the name of the corresponding element may vary depending on a type of the electronic device. The electronic device according to various embodiments of the present disclosure may include at least one of the aforementioned elements or may further include other additional elements, or some of the aforementioned elements may be omitted. Also, a few component elements of an electronic device according to various embodiments of the present disclosure may be coupled to form a single entity, and may equivalently execute functions of the corresponding component elements which are not coupled.

The term “module” as used in various embodiments of the present disclosure may refer to, for example, a “unit” including one of hardware, software, and firmware, or a combination of two or more of the hardware, software, and firmware. The term “module” may be interchangeable with another term, such as a unit, a logic, a logical block, a component, or a circuit. The term “module” may be a minimum unit of an integrated component element or a part thereof. The term “module” may be a minimum unit for performing one or more functions or a part thereof. The term “module” may be mechanically or electronically implemented. For example, the term “module” according to various embodiments of the present disclosure may include at least one of an Application-Specific Integrated Circuit (ASIC) chip, a Field-Programmable Gate Arrays (FPGAs), and a programmable-logic device for performing operations which have been known or are to be developed hereafter.

A blood flow rate of a peripheral blood vessel changes as the contraction and relaxation of a heart is repeated, and a volume of a blood vessel is changed. In connection with this, photoplethysmography (PPG), according to various embodiments of the present disclosure, is a technology for measuring a transmission quantity of light by using an optical sensor and showing a heart beat in the form of a waveform (i.e., a PPG signal). A heart rate or oxygen saturation may be determined based on information obtained through the PPG signal.

In order to perform PPG by the electronic device 200, a finger of a measurement target person is in contact with a sensor part, and light emitted from a light source (not illustrated) may pass through the various media of the finger, be diffused and dispersed, and be widely spread, and some of the light may be detected through an optical sensor (not illustrated), for example, a photodiode. In the meantime, the PPG, according to various embodiments of the present disclosure, may be performed at a finger, a toe, a wrist, an ear lobe, a forehead, etc., of a measurement target person. However, the finger is more sensitive to the response of the automatic nervous system than other regions, so that the signals may be mainly measured at a finger.

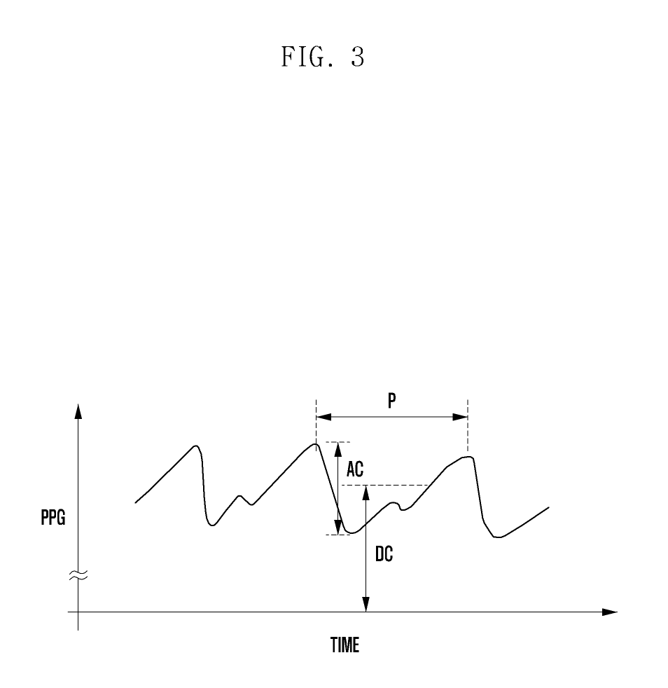

FIG. 3 illustrates a waveform of a PPG signal according to an embodiment of the present disclosure.

Referring to FIG. 3, the PPG signal may be measured by using a change in an optical characteristic of a finger. More specifically, the amount of blood is increased in a finger during a systolic phase of the heart, so that the finger becomes slightly dark. Further, the amount of blood is decreased in the finger during a diastolic phase of the heart, so that the finger becomes brighter or less dark. At the systolic phase, the amount of light entering the photodiode is small, but at the diastolic phase, the amount of light entering the photodiode is increased. The PPG signal may be divided into an AC component and a DC component. The AC component, which is a pulsatile component, expresses a pulsatile morphology of a blood vessel, which is synchronized with a heartbeat to be changed. Further, the DC component, which is a non-pulsatile component, is a signal measured by using light reflected by parts having a uniform volume, such as a bone, a pigment of a skin, a human tissue, and the like, except for the signal according to a change in a volume of a blood vessel (e.g., an artery). Referring to FIG. 3, it can be seen that in the PPG signal, the systolic phase and the diastolic phase are repeated with a period P.

The PPG signal measured according to the various embodiments of the present disclosure may be used for, for example, measurement of a heart rate (i.e., a pulse). As an embodiment of the present disclosure, the heart rate may be measured by finding a minimum brightness point of the PPG signal and analyzing a change quantity, measuring only a change at a specific threshold value or more, and measuring a frequency through frequency conversion. As a result, it is possible to recognize the number of times of generation of a pulse per second, and to measure a heart rate by using the recognized number of times of generation of the pulse per second.

According to an embodiment of the present disclosure, the electronic device 200 may include a PPG measurement module.

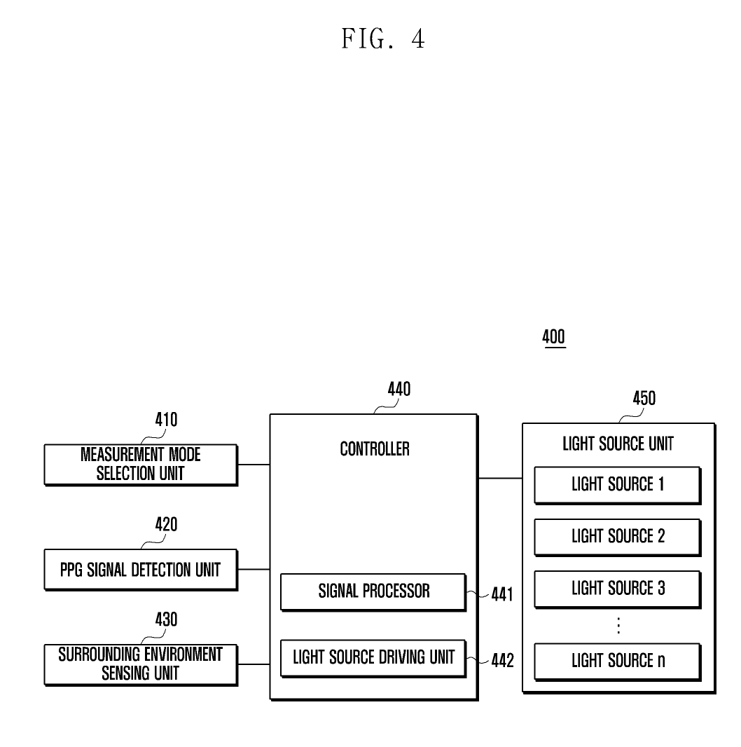

FIG. 4 is a diagram schematically illustrating a PPG measurement module of the electronic device according to an embodiment of the present disclosure.

The PPG measurement module according to an embodiment of the present disclosure may include a plurality of light sources having different wavelength characteristics. For example, the PPG measurement module may include a plurality of light sources having different wavelength bands, such as green, red, and infrared wavelength bands. According to the PPG measurement module, it is possible to measure a PPG signal by selecting a light source appropriate to a measurement object or a measurement environment.

Referring to FIG. 4, the PPG measurement module 400 may include a measurement mode selection unit 410, a PPG signal detection unit 420, a surrounding environment sensing unit 430, a controller 440, and a light source unit 450.

The measurement mode selection unit 410 may select a measurement mode according to an object of the PPG signal measurement, and transmit the selected measurement mode to the controller 440. The measurement mode may be selected by initial setting, by a user's input, etc. In addition, the measurement mode may be determined by various methods. The measurement mode may include, for example, a heart rate measurement mode, an oxygen saturation measurement mode, and the like.

The PPG signal detection unit 420 may detect light, which is irradiated from any light source included in the light source unit 450 and passes through or is reflected from a tissue of a measurement target person. The detected result may be transmitted to the controller 440. For example, the PPG signal detection unit 420 may include an optical sensor detecting light.

The surrounding environment sensing unit 430 may collect information about a surrounding environment where the PPG is performed by using various sensors, and transmit the collected information to the controller 440. For example, the surrounding environment sensing unit 430 may sense a surrounding temperature or a temperature of a measurement target person by using a temperature sensor, sense light information about a surrounding environment by using a separate optical sensor, and sense a movement of the electronic device 200 or a movement of a measurement target person by using a motion sensor. Further, the surrounding environment sensing unit 430 may sense a skin color of a measurement target person by using an image sensor. The surrounding environment sensing unit 430 may include the sensor module 240 illustrated in FIG. 2.

The controller 440 may determine a characteristic of a light source based on at least one of outputs of the measurement mode selection unit 410, the PPG signal detection unit 420, and the surrounding environment sensing unit 430. An example of the light source may include an LED, and the controller 440 may determine a characteristic of a light source by selecting any one LED among a plurality of LEDs. However, the present disclosure is not limited thereto, and the controller 440 may determine a characteristic of the light source by various methods. Further, the controller 440 may detect a PPG signal based on the output of the PPG signal detection unit 420 and calculate the detected PPG signal to measure a biometric numeral value, such as a heart rate or oxygen saturation, related to health information about a measurement target person.

For example, the controller 440 may include a signal processor 441 and a light source driving unit 442.

The signal processor 441 may serve to receive outputs of the measurement mode selection unit 410, the PPG signal detection unit 420, and the surrounding environment sensing unit 430, and process a signal.

For example, the signal processor 441 may detect a PPG signal from an output signal of the PPG signal detection unit 420. Further, a biometric numerical value conforming to the measurement object may be determined by calculating the detected PPG signal. The determined biometric numerical value may be visually or audibly output through a display unit, so as to allow a user to recognize the induced biometric numerical value.

Further, the signal processor 441 may deduce information on a currently set measurement mode from the output signal of the measurement mode selection unit 410. For example, the signal processor 441 may deduce information about whether the currently set measurement mode is a heart rate measurement mode or an oxygen saturation measurement mode.

Further, the signal processor 441 may deduce surrounding environment information from the output signal of the surrounding environment sensing unit 430. For example, the signal processor 441 may deduce at least one piece of information from among current surrounding temperature information, temperature information about a measurement target person, light information about a surrounding environment, movement information about the electronic device 200 or a measurement target person, or skin color information about a measurement target person.

The light source driving unit 442 may select and drive any one of the light sources having different wavelength characteristics based on at least one of the detected PPG signal, the measurement mode information, and the surrounding environment information. In addition, the light source driving unit 442 may adjust a light quantity characteristic of the selected light source based on at least one of the detected PPG signal, the measurement mode information, and the surrounding environment information.

Various embodiments of the present disclosure, in which the light source driving unit 442 selects a characteristic of a light source, will be described below.

For example, in the case where the measurement mode information indicates the oxygen saturation measurement mode, light sources having red and infrared wavelength characteristics may be selected in order to measure oxygen saturation. In general, when red light passes through oxyhemoglobin, a light absorption rate is lower than that of dioxyhemoglobin, and when infrared ray passes through oxyhemoglobin, a light absorption rate of oxyhemoglobin is greater than that of dioxyhemoglobin. The difference may be used as a basic principle for determining oxygen saturation. Accordingly, in order to measure oxygen saturation, two light sources having the red and infrared wavelength characteristics may be selected in the light source unit 450.

In the case of the heart beat measurement mode, a pulsatile component of the PPG signal is analyzed, so that a wavelength characteristic may not be separately selected.

In the case where the surrounding environment information includes the surrounding temperature or the temperature information about the measurement target person, and a corresponding temperature is detected to be lower than a reference value, the light source driving unit 442 may select a light source having a comparatively long wavelength characteristic, for example, the light source having an infrared characteristic. The light source having a comparatively long wavelength characteristic is resistant to cold, thereby being used. According to an embodiment of the present disclosure, a light source having a wavelength characteristic corresponding to a level of the measured temperature information may be selected based on the measured temperature information.

In the case where the surrounding environment information includes light information about the surrounding environment, it may be determined whether light of the surrounding environment may causes noise during the measurement of the PPG signal. If it is determined that the light of the surrounding environment may cause noise, a wavelength of the light of the surrounding environment may be determined, and a light source having a wavelength, other than the determined wavelength of the surrounding environment, may be selected.

Further, in the case where the surrounding environment information includes information on a movement of the electronic device 200 or a movement of a measurement target person, and it is detected that the movement is generated to have a reference value or more based on the corresponding information, the light source driving unit 442 may select a light source having a comparatively short wavelength characteristic, for example, a light source having a green characteristic. The use of a light source having a comparatively short wavelength characteristic is advantageous to a movement, thereby being used. According to an embodiment of the present disclosure, a light source having a wavelength characteristic corresponding to a level of the measured movement information may be selected based on the measured movement information.

Further, in the case where the surrounding environment information includes skin color information, and it is detected that the skin color is darker than a reference color, selection of a light source having a short wavelength characteristic, for example, a light source having a green characteristic, may be rejected. The reason is that light having a green characteristic is considerably influenced by brightness. Accordingly, in the case where it is detected that the skin color is a relatively bright color, the light source driving unit 442 may select a light source having a short wavelength characteristic, and in the case where it is detected that the skin color is a relatively dark color, the light source driving unit 442 may select a light source having a relatively long wavelength characteristic. According to an embodiment of the present disclosure, a light source having a wavelength characteristic corresponding to a level of the measured skin color information may be selected based on the measured movement information.

According to an embodiment of the present disclosure, a wavelength characteristic of the light source may be determined by considering a combination of the temperature information, the movement information, and the skin color information, as well as each of the temperature information, the movement information, and the skin color information.

The detected PPG signal may have a different characteristic for each measurement target person. The reason is that a factor influencing the PPG may have a different characteristic for each person. Accordingly, a wavelength characteristic of the light source capable of generating a preferred PPG signal for each person may be different. When the light source driving unit 442 according to an embodiment of the present disclosure senses the detected PPG signal, and determines that the detected PPG signal is distorted to be inappropriate to the analysis, the light source driving unit 442 may select a light source having a different wavelength characteristic. The light source driving unit 442 may continuously select a light source having a different wavelength characteristic until the preferred PPG signal is obtained.

In addition, the light source driving unit 442 may adjust a light quantity of the selected light source based on the detected PPG signal.

In the case where the detected PPG signal is not included in an appropriate section in which the PPG is easily performed, for example, a size of the PPG signal is excessively small, so that the PPG signal is detected to be included in a section, in which a ratio with respect to noise, that is a Signal-to-Noise Ratio (SNR), is excessively low, or a size of the PPG signal is excessively large, so that the PPG signal is detected to be included in a signal saturation region, a quantity of light of the light source may be adjusted so that the PPG signal may be generated in an appropriate section.

In the case where the PPG signal is detected to be included in the section in which the SNR is very low, that is, it is detected that the size of the PPG signal is lower than a reference value, the quantity of light of the selected light source may be increased. However, in the case where the PPG signal is in a saturation state, the quantity of light of the selected light source may be decreased.

The light source unit 450 is an example of a plurality of light sources having various wavelength bands, and according to an embodiment of the present disclosure, the light source unit 450 may include light sources (a light source 1 to a light source n) having various wavelength characteristics.

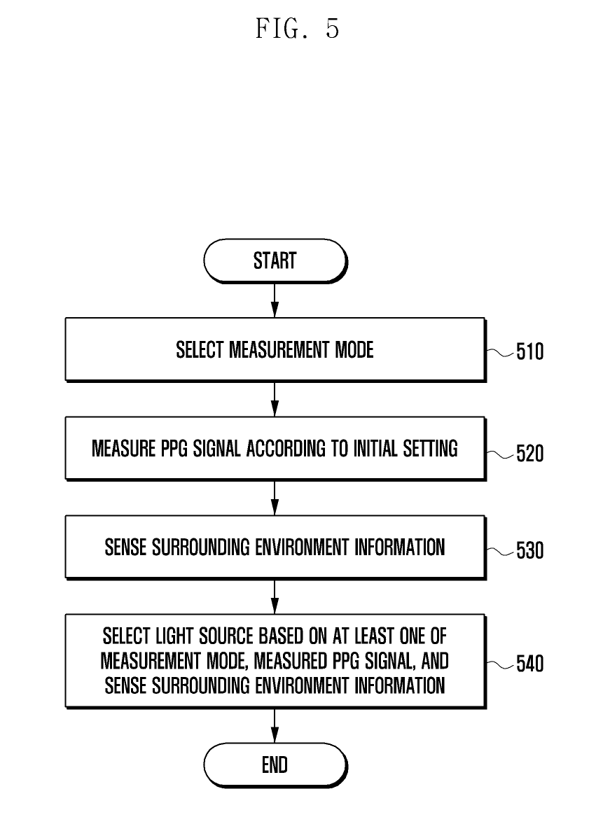

FIG. 5 is a flowchart schematically illustrating a PPG method according to an embodiment of the present disclosure.

Referring to FIG. 5, the electronic device 200 may select a measurement mode according to an object of the PPG in operation 510. Further, a PPG signal may be measured according to an initial setting in operation 520. In this case, the initial setting of the measurement of the PPG signal may be a default, and may be determined according to the measurement mode set in operation 510. The electronic device 200 may sense surrounding environment information in operation 530. The sensed surrounding environment information may include the temperature, movement, skin color information, and the like as described above. The electronic device 200 may determine a light source characteristic by using at least one of the selected measurement mode, the measured PPG signal, and the sensed surrounding environment information in operation 540. The determination of the light source characteristic may include, for example, selection of one or more of the plurality of light sources having various wavelength characteristics, and adjustment of a light quantity characteristic of the selected light source.

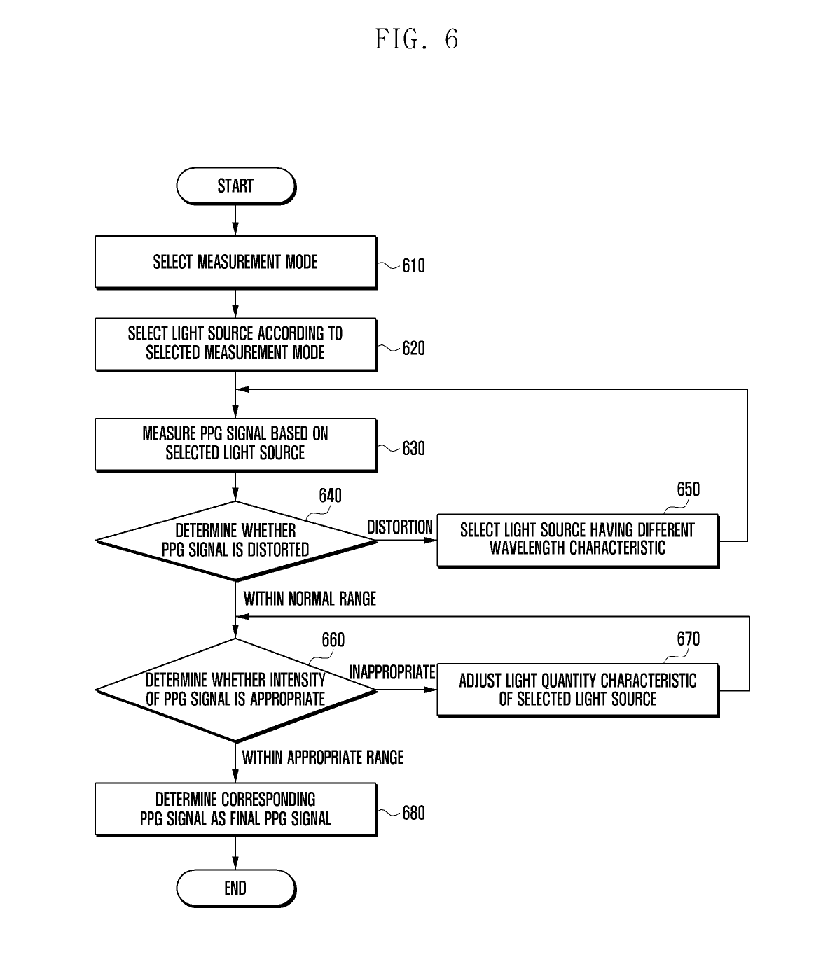

FIG. 6 is a flowchart illustrating an example, in which a measurement mode and a measured PPG signal are considered in the PPG method according to an embodiment of the present disclosure.

Referring to FIG. 6, the electronic device 200 may select a measurement mode according to an object of the PPG in operation 610. Further, the electronic device 200 may select a light source having a specific wavelength characteristic according to the selected measurement mode in operation 620. Then, the electronic device 200 may measure a PPG signal based on the selected light source in operation 630.

The electronic device 200 may determine whether the measured PPG signal is distorted in operation 640. Whether the measured PPG signal is distorted may include determining whether the measured PPG signal is appropriate to an analysis. In the case where a distorted PPG signal, which does not belong to a threshold value, is detected in a corresponding wavelength characteristic, the electronic device 200 may select a light source having a different wavelength characteristic, and measure the PPG signal based on the corresponding light source again in operation 650. On the other hand, when the PPG signal within a normal range based on a threshold value is detected in the corresponding wavelength characteristic, the electronic device 200 may determine whether an intensity of the PPG signal is appropriate in operation 660. Whether the intensity of the PPG signal is appropriate may be determined by a method of detecting whether an SNR is low and the PPG signal is in a saturation state.

When it is determined that the intensity of the PPG signal is not appropriate, a light quantity characteristic of the selected light source may be adjusted in operation 670. For example, when it is determined that the SNR of the PPG signal is low, the light quantity of the selected light source may be increased, and when it is determined that the PPG signal is in the saturation state, the light quantity of the selected light source may be decreased.

On the other hand, when it is determined that the intensity of the PPG signal is within an appropriate range, the electronic device 200 may determine the corresponding PPG signal as a final PPG signal in operation 680. The electronic device 200 may calculate and induce a biometric numerical value in accordance with an object of the PPG by using the determined final PPG signal.

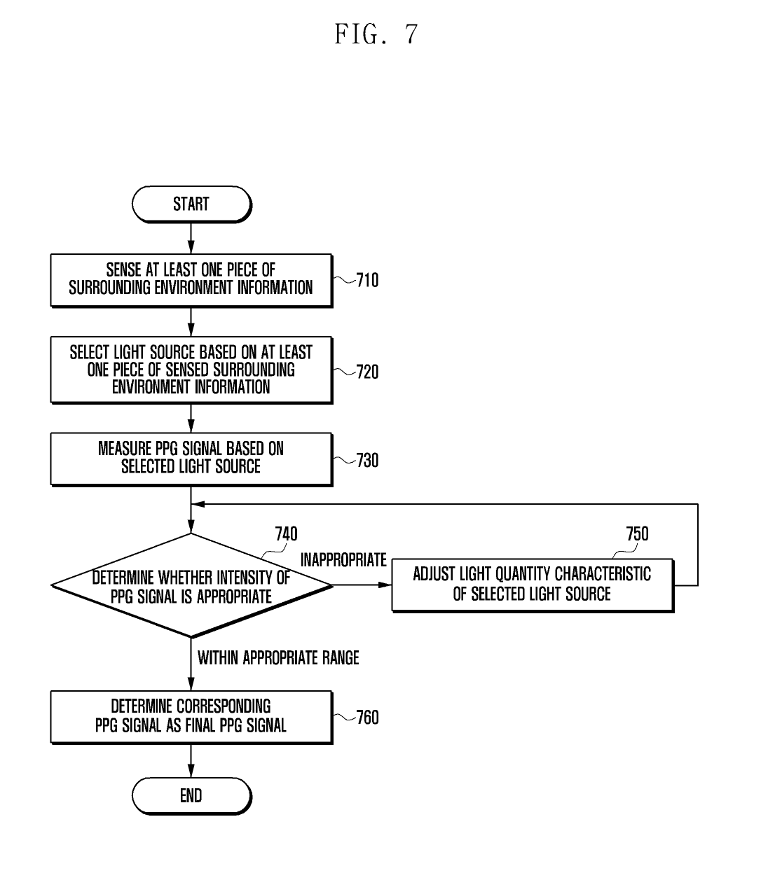

FIG. 7 is a flowchart illustrating an example, in which the neighboring environment information is considered in the PPG method according to an embodiment of the present disclosure.

Referring to FIG. 7, the electronic device 200 may sense at least one piece of information among various surrounding environment information, such as temperature information, surrounding light information, movement information, and skin color information, in operation 710. Further, the electronic device 200 may select a light source having an appropriate wavelength characteristic based on the sensed surrounding environment information in operation 720. The wavelength characteristic appropriate to the surrounding environment information has been described with reference to FIG. 4. The electronic device 200 may measure a PPG signal based on the selected light source in operation 730.

The electronic device 200 may determine whether an intensity of the measured PPG signal is appropriate in operation 740. When it is determined that the intensity of the PPG signal is not appropriate, a light quantity characteristic of the selected light source may be adjusted in operation 750. For example, when it is determined that the SNR of the PPG signal is low, the light quantity of the selected light source may be increased, and when it is determined that the PPG signal is in the saturation state, the light quantity of the selected light source may be decreased.

On the other hand, when it is determined that the intensity of the PPG signal is within an appropriate range, the electronic device 200 may determine the corresponding PPG signal as a final PPG signal in operation 760. The electronic device 200 may calculate and induce a biometric numerical value in accordance with an object of the PPG by using the determined final PPG signal.

Those skilled in the art will appreciate that the various embodiments of the present disclosure are not limited to the embodiments of FIGS. 6 and 7, but may include any embodiment in which a characteristic of the light source is determined by considering the various measurement modes, the PPG signal, the surrounding environment information, and the like.

According to various embodiments of the present disclosure, at least part of a device (for example, modules or functions thereof) or a method (for example, operations) according to the various embodiments of the present disclosure may be embodied by, for example, an instruction stored in a non-transitory computer readable storage medium provided in a form of a programming module. When the instructions are performed by at least one processor (e.g., the processor 120), the at least one processor may perform functions corresponding to the instructions. The computer readable storage media may be, for example, the memory 130. At least a part of the programming module may be implemented (for example, executed) by, for example, the processor 120. At least a part of the programming module may include, for example, a module, a program, a routine, a set of instructions, or a process for performing at least one function.

The computer-readable recording medium may include magnetic media such as a hard disk, a floppy disk, and a magnetic tape, optical media such as a Compact Disc ROM (CD-ROM) and a DVD, magneto-optical media such as a floptical disk, and hardware devices specially configured to store and perform a program instruction (for example, programming module), such as a ROM, a RAM, a flash memory and the like. Further, the program commands may include high class language codes that can be executed in a computer by using an interpreter, as well as machine language codes that are made by a compiler. The aforementioned hardware device may be configured to operate as one or more software modules in order to perform the operation of various embodiments of the present disclosure, and vice versa.

A programming module according to the present disclosure may include at least one of the described component elements, one or more of the component elements may be omitted, or one or more additional component elements may be included. Operations executed by a module, a programming module, or other component elements according to various embodiments of the present disclosure may be executed sequentially, in parallel, repeatedly, or in a heuristic manner. Also, a few operations may be executed based on a different order, may be omitted, or may additionally include another operation.

While the present disclosure has been shown and described with reference to various embodiments thereof, it will be understood by those skilled in the art that various changes in form and details may be made therein without departing from the spirit and scope of the present disclosure as defined by the appended claims and their equivalents.

What is claimed is:

1. A photoplethysmography (PPG) method comprising:
performing at least one of PPG signal measurement and surrounding environment information sensing; and
determining a characteristic of a light source for measuring a PPG signal based on at least one of the measured PPG signal or the sensed surrounding environment information.

2. The PPG method of claim 1, wherein the surrounding environment information sensing includes sensing at least one of a skin temperature of a user, a temperature of a surrounding environment, light of a surrounding environment, a movement of a user, or a skin color of a user.

3. The PPG method of claim 1, wherein the PPG signal measurement is performed based on a characteristic of a selected light source in response to at least one of initial setting, a measurement mode, and the sensed surrounding environment information.

4. The PPG method of claim 3, further comprising:
selecting one of a heart rate measurement mode and an oxygen saturation measurement mode as the measurement mode.

5. The PPG method of claim 1, wherein the determining of the characteristic of the light source includes determining at least one of a wavelength characteristic and a light quantity characteristic of the light source.

6. The PPG method of claim 5, wherein the determining of the characteristic of the light source includes:
determining whether the measured PPG signal is distorted; and
adjusting a wavelength characteristic of the light source when it is determined that the measured PPG signal is distorted.

7. The PPG method of claim 5, wherein the determining of the characteristic of the light source includes:
determining whether an intensity of the measured PPG signal is appropriate; and
adjusting a light quantity characteristic of the light source when it is determined that the intensity of the measured PPG signal is not appropriate.

8. The PPG method of claim 5, further comprising:
determining whether the measured PPG signal is distorted;
adjusting a wavelength characteristic of the light source when it is determined that the measured PPG signal is distorted;
determining whether an intensity of the measured PPG signal is appropriate when it is determined that the measured PPG signal is within a normal range; and
adjusting a light quantity characteristic of the light source when it is determined that the intensity of the measured PPG signal is not appropriate.

9. The PPG method of claim 5, further comprising
selecting a corresponding light source according to the determined wavelength characteristic of the light source.

10. The PPG method of claim 9, further comprising:
adjusting a light quantity of the selected light source according to the determined wavelength characteristic of the light source.

11. An electronic device comprising:
a plurality of light sources having different wavelength characteristics;
a photoplethysmography (PPG) signal detection unit configured to detect light, which is irradiated from at least one of the plurality of light sources, and passes through or is reflected from a user; and
a controller configured to detect a PPG signal from an output signal of the PPG signal detection unit and to select at least one of the plurality of light sources based on the detected PPG signal.

12. The electronic device of claim 11, further comprising:
a measurement mode selection unit configured to select any one of a heart rate measurement mode and an oxygen saturation measurement mode,
wherein the controller selects at least one of the plurality of light sources based on at least one of the detected PPG signal and a measurement mode selected by the measurement mode selection unit.

13. The electronic device of claim 11, further comprising:
a surrounding environment sensing unit configured to sense at least one of a skin temperature of a user, a temperature of a surrounding environment, light of a surrounding environment, a movement of a user, or a skin color of a user,
wherein the controller selects at least one of the plurality of light sources based on at least one of the detected PPG signal, the measurement mode, and the surrounding environment information sensed by the surrounding environment sensing unit.

14. The electronic device of claim 13, wherein the controller determines whether the detected PPG signal is distorted, and selects a light source having another wavelength characteristic when it is determined that the detected PPG signal is distorted.

15. The electronic device of claim 13, wherein the controller determines whether an intensity of the detected PPG signal is appropriate, and adjusts a light quantity of the selected light source when it is determined that the intensity of the detected PPG signal is not appropriate.

16. The electronic device of claim 13, wherein the controller determines whether the measured PPG signal is distorted, and selects a light source having another wavelength characteristic when it is determined that the detected PPG signal is distorted.

17. The electronic device of claim 16, wherein the controller determines whether an intensity of the detected PPG signal is appropriate when it is determined that the detected PPG signal is within a normal range, and adjusts a light quantity of the selected light source when it is determined that the intensity of the measured PPG signal is not appropriate.

18. A computer readable recording medium, in which a program for executing an operation performing at least one of photoplethysmography (PPG) signal measurement and surrounding environment information sensing, and determining a characteristic of a light source for the PPG signal measurement based on at least one of the measured PPG signal and the sensed surrounding environment information is recorded.
